(19) United States
(12) Patent Application Publication
(10) Pub. No.: US 2023/0172913 A1
(43) Pub. Date: Jun. 8, 2023

(54) COMPOUNDS FOR USE IN THE REACTIVATION OF HIV IN LATENT HIV-INFECTED CELLS

(71) Applicants: CENTRE NATIONAL DE LA RECHERCHE SCIENTIFIQUE, PARIS (FR); UNIVERSITE DE MONTPELLIER, MONTPELLIER (FR)

(72) Inventors: BRUNO BEAUMELLE, SAINT DREZERY (FR); LAURENT CHALOIN, PRADES LE LEZ (FR)

(21) Appl. No.: 17/925,618

(22) PCT Filed: May 18, 2021
(86) PCT No.: PCT/EP2021/063206

§ 371 Date: Nov. 16, 2022

(30) Foreign Application Priority Data
May 20, 2020 (FR) FR2005250

(51) Int. Cl. A61K 31/433; A61K 45/06; A61P 31/18; A61K 31/4184; A61K 31/52; A61K 31/428; A61K 31/437; A61K 31/4418; A61K 31/517; A61K 31/498; A61K 31/519; A61K 31/655; A61K 31/423

(52) U.S. Cl. CPC A61K 31/433; A61K 45/06; A61P 31/18; A61K 31/4184; A61K 31/52; A61K 31/428; A61K 31/437; A61K 31/4418; A61K 31/517; A61K 31/498; A61K 31/519; A61K 31/655; A61K 31/423

(57) ABSTRACT

The present invention relates to compounds capable of binding to the Tat-TAR complex in latent HIV-infected cells, so as to transactivate the Tat protein and promote the lifting of HIV latency in said cells.

### TECHNICAL FIELD

The present invention relates to the treatment of viral diseases, and more particularly to the treatment of HIV-related diseases. More specifically, the invention relates to compounds capable of reactivating HIV in latent HIV-infected cells.

### TECHNOLOGICAL BACKGROUND

The human immunodeficiency virus, or HIV, is a retrovirus of the lentivirus genus that infects humans and is responsible for acquired immunodeficiency syndrome (AIDS). The global spread of HIV and the extremely high number of people infected with the virus have made AIDS a global health priority.

HIV infection is a chronic infection with continuous viral replication leading to decreased T CD4 lymphocyte counts and immunosuppression. Viral replication can be reduced by antiretroviral medications (ARTs). Antiretroviral treatments (ARTs) use small molecules capable of inhibiting HIV viral replication and grouped into classes according to their target. The reduction in viral replication is followed by an increase in T CD4 lymphocytes.

Although ARTs effectively suppress HIV-1 viral replication by blocking various stages of the virus life cycle, ARTs do not cure the infection due to the existence of long-lived reservoirs. These are mainly quiescent T-CD4⁺ lymphocytes that contain an integrated HIV-1 provirus but do not produce virus. Latent protein expression is limited. The infected cell is therefore undifferentiated from normal cells and is not targeted by either ART or the immune system. Latency is a non-productive but reversible stage of viral infection. It is these latent cells that will allow viral rebound when ART is interrupted. Thus, the viral load becomes detectable again in the majority of infected individuals after 2 weeks of interruption of ART treatment. Latent infected T lymphocytes are rare (1-100 per million T-CD4⁺ cells according to viral growth assays) but they have an extremely long half-life (44 months). These latent cells therefore constitute a major obstacle to viral eradication. Furthermore, this reservoir of latent cells is set up dramatically early after infection: a patient infected ten days previously already has nearly 200 latent cells in the body and therefore cannot be “cured” by ARTs.

One of the strategies aiming at eradicating this HIV-1 reservoir requires the activation of these latent infected cells (“Shock” phase), so that they can be killed by the virus or recognized by immune effectors (“Kill” phase). This is the “shock-and-kill” paradigm.

The activation of HIV-1 in latent infected cells requires the use of latency lifting or reversing agents (LRAs). Most of the available LRAs target cellular proteins such as histone deacetylases (HDACs), protein kinase C, methyl transferases or the transcription elongation factor P-TEFb. Although these LRAs can activate viral production in cell lines or primary cells harboring latent viruses, especially when used in combination, none of them has so far demonstrated a significant effect on the size of the viral reservoir in patients. Indeed, these LRAs target cellular proteins and have many side effects. In particular, they inhibit the cytotoxic function of CD8 cells, whereas this is crucial for the “Kill” phase. This effect is probably responsible for the inability of these molecules to decrease the size of the reservoir in patients.

Thus, there remains a need for safe compounds capable of effectively reactivating HIV in latent cells of HIV-infected patients.

### SUMMARY OF THE INVENTION

In this context, the inventors have identified new LRA compounds no longer targeting cellular proteins but a viral protein. More specifically, the compounds according to the invention activate the Tat (“Trans-Activator of Transcription”) protein of the HIV, which by interacting with the TAR domain (“Trans-Activation Responsive element”) of the viral RNA sequences, allows the transcription of viral genes. The TAR domain is composed of the first 57 nucleotides common to all HIV RNAs. Tat is a key viral protein that is expressed at low levels in latent cell lines. The compounds according to the invention have a strong affinity for the Tat-TAR transcription complex, and promote the transcriptional activity of this protein, thus allowing to remove the latency of HIV in HIV-infected cells.

The invention therefore relates to a compound according to formula (I), or a salt thereof,

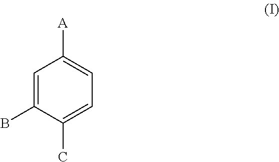

wherein

A represents a heteroaryl optionally substituted by at least one halogen, and

B and C represent, independently of each other, a radical selected from a hydrogen, a C₁-C₆ alkyl, a C₁-C₆ alkoxy, and a group according to formula (II),

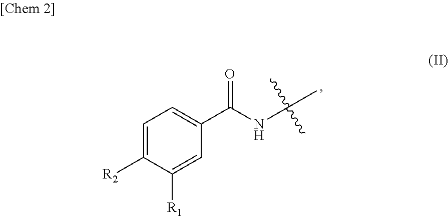

wherein R₁ and R₂ represent, independently of each other, a radical selected from

a hydrogen,

a C₁-C₃ alkyl,

a C₁-C₃ alkoxy,

a group according to the formula —Y—[W]n-Z, wherein

Y is a radical selected from O, N, S and C, preferably a radical selected from O, N and C

W is a radical selected from a C₁-C₆ alkyl, a C₁-C₆ alkylene and a C₂-C₆ alkenyl,

n is equal to 0 or 1, preferably equal to 1,

Z represents a phenyl, a pyrrole, a naphthalene, an aryl, an imidazole, a naphthyl or a phenanthrene, preferably a phenyl, optionally substituted by at least one radical selected from a C₁-C₃ alkyl, a C₁-C₃ alkoxy and a halogen, preferably substituted by one halogen, or two halogens,

wherein

when C is hydrogen, methyl or methoxy, B is a group according to formula (II), and conversely when B is hydrogen, methyl or methoxy, C is a group according to formula (II), and

when R₁ is hydrogen, C₁-C₃ alkyl or C₁-C₃ alkoxy, R₂ is the group —Y—[W]n-Z, and conversely when R₂ is hydrogen, C₁-C₃ alkyl or C₁-C₃ alkoxy, R₁ is the group —Y—[W]n-Z, for use in a method for treating HIV infection in a subject with HIV and/or for use in a method for treating HIV latency in latent HIV-infected cells in a subject with HIV.

A more particular object of the invention is a compound according to formula (I) capable of activating the Tat protein, or a salt thereof, for its use in a method for treating an HIV infection in a subject with HIV and/or for its use in a method for treating HIV latency in latent HIV-infected cells in an HIV-infected subject.

The object of the invention is in particular a compound according to formula (I), having a strong affinity for the Tat-TAR transcription complex, and promoting the transcriptional activity of this protein, or a salt thereof, for its use in a method for treating HIV infection in an HIV-infected subject and/or for use in a method for treating HIV latency in latent HIV-infected cells in an HIV-infected subject.

The invention also relates to the use of a compound according to formula (I) capable of activating the Tat protein, or a salt thereof, for the preparation of a medication for treating an HIV infection in a subject with HIV and/or for the preparation of a medication for treating HIV latency in latent HIV-infected cells in a subject with HIV.

A particular object of the invention is the use of a compound according to formula (I), having a strong affinity for the Tat-TAR transcription complex, and promoting the transcriptional activity of this protein, or a salt thereof, for the preparation of a medication for treating HIV infection in a subject with HIV and/or for the preparation of a medication for treating HIV latency in latent HIV-infected cells in a subject with HIV.

In one embodiment, A is selected from benzimidazole, imidazo[2,1-b]-1,3,4-thiadiazole, benzodiazole, benzothiazole, benzoxazolone, purine, pyridine, oxazolone, quinazoline and a quinoxaline, optionally substituted by at least one halogen.

In one embodiment, B and C independently represent a radical selected from hydrogen and a group according to formula (II).

In one embodiment, B and C represent, independently of each other, a radical selected from a C_{1-C6} alkoxy, preferably a C₁-C₃ alkoxy, more preferably a methoxy or an ethoxy, and a group according to formula (II).

In one embodiment, B and C represent, independently of each other, a radical selected from a C_{1-C6} alkyl, preferably a methyl or an ethyl, and a group according to formula (II).

In one embodiment, the group —Y—[W]n-Z represents a naphthalenoxy, a pyrolmethoxy, a phenyloxy, or a benzene, preferentially a benzyloxy, optionally substituted by one or more radicals selected from halogen and C₁-C₃ alkyl.

In one embodiment, C is hydrogen, methyl or methoxy, and B is a group according to formula (II), wherein R₁ is methoxy or hydrogen and R₂ is benzyloxy optionally substituted by a halogen, preferably a chlorine.

In another embodiment, B is hydrogen, methyl or methoxy, and C is a group according to formula (II), wherein R₁ is methoxy or hydrogen and R₂ is benzyloxy optionally substituted by a halogen, preferably a chlorine.

In one embodiment, C is hydrogen, and B is a group according to formula (II), wherein R₁ is hydrogen and R₂ is benzyloxy optionally substituted by at least one halogen, preferably one chlorine, more preferably substituted by two chlorines.

In another embodiment, B is hydrogen, and C is a group according to formula (II), wherein R₁ is hydrogen and R₂ is benzyloxy optionally substituted by at least one halogen, preferably chlorine, more preferably substituted by two chlorines.

In one embodiment, C is hydrogen, and B is a group according to formula (II), wherein R₁ is methoxy and R₂ is benzyloxy optionally substituted by at least one halogen, preferably chlorine, more preferably substituted by two chlorines.

In another embodiment, B is hydrogen, and C is a group according to formula (II), wherein R₁ is methoxy and R₂ is benzyloxy optionally substituted by at least one halogen, preferably chlorine, more preferably substituted by two chlorines.

In one embodiment, C is hydrogen, and B is a group according to formula (II), wherein R₁ is ethyloxy and R₂ is benzyloxy optionally substituted by at least one halogen, preferably chlorine, more preferably substituted by two chlorines.

In another embodiment, B is hydrogen, and C is a group according to formula (II), wherein R₁ is ethyloxy and R₂ is benzyloxy optionally substituted by at least one halogen, preferably chlorine, more preferably substituted by two chlorines.

In one embodiment, C is methyl, and B is a group according to formula (II), wherein R₁ is hydrogen and R₂ is benzyloxy optionally substituted by at least one halogen, preferably chlorine, more preferably substituted by two chlorines.

In another embodiment, B is methyl, and C is a group according to formula (II), wherein R₁ is hydrogen and R₂ is benzyloxy optionally substituted by at least one halogen, preferably chlorine, more preferably substituted by two chlorines.

In one embodiment, C is methoxy, and B is a group according to formula (II), wherein R₁ is hydrogen and R₂ is benzyloxy optionally substituted by at least one halogen, preferably chlorine, more preferably substituted by two chlorines.

In another embodiment, B is methoxy, and C is a group according to formula (II), wherein R₁ is hydrogen and R₂ is benzyloxy optionally substituted by at least one halogen, preferably chlorine, more preferably substituted by two chlorines.

In one embodiment, C is methyl, and B is a group according to formula (II), wherein R₁ is methoxy and R₂ is benzyloxy optionally substituted by at least one halogen, preferably chlorine, more preferably substituted by two chlorines.

In another embodiment, B is methyl, and C is a group according to formula (II), wherein R₁ is methoxy and R₂ is benzyloxy optionally substituted by at least one halogen, preferably chlorine, more preferably substituted by two chlorines.

In a particular embodiment:
A represents a benzimidazole optionally substituted by a halogen (preferably a chlorine), and
B and C represent, independently of each other, a radical selected from:

a hydrogen,

a C₁-C₆ alkyl,

a C₁-C₆ alkoxy, and

a group according to formula (II), wherein R₁ and R₂ represent, independently of each other, a radical selected from:

a hydrogen,

a C₁-C₂ alkoxy,

a group according to the formula —Y—[W]n-Z,

wherein

Y is a radical selected from O, N and C (preferably O);

W is a radical selected from a methyl (—CH₂—), a linear ethyl (—CH₂—CH₂—) and an ethenyl (—CH═CH—),

n is equal to 1,

Z represents a phenyl, a pyrrole or a naphthalene, aryl, an imidazole, naphthyl, or a phenanthrene (preferably a phenyl), optionally substituted by at least one radical selected from a C₁-C₃ alkyl, a C₁-C₃ alkoxy and a halogen.

In another particular embodiment:
A is an imidazo[2,1-b]-1,3,4-thiadiazole,
B and C represent, independently of each other, a radical selected from:

a hydrogen,

a C₁-C₆ alkoxy, and

a group according to formula (II) wherein R₁ and R₂ represent independently of each other, a radical selected from

a hydrogen,

a C₁-C₂ alkoxy,

a group according to the formula —Y—[W]n-Z,

wherein

Y is a radical selected from O, N, and C (preferably O);

W is C₁-C₂ alkyl,

n is equal to 1;

Z represents a phenyl, a pyrrole or a naphthalene, aryl, an imidazole, naphthyl, or a phenanthrene (preferably a phenyl) optionally substituted by at least one radical selected from a C₁-C₃ alkyl, a C₁-C₃ alkoxy and a halogen,

provided that when R₁ is methoxy, R₂ is a group of formula (II) wherein Z is phenyl substituted by a halogen (preferably chlorine or fluorine).

In a particular embodiment, A is benzoxazole, benzothiazole, pyridine, quinazoline, purine, imidazo[1,2a]pyrimidine, or thiazolo[5,4-b]pyridine. In such a particular embodiment, it is preferred that:

B and C independently represent a radical selected from:

a hydrogen,

a C₁-C₆ alkyl (preferably a methyl),

a C₁-C₆ alkoxy (preferably a methoxy), and

a group according to formula (II), wherein R₁ and R₂ represent, independently of each other, a radical selected from:

a hydrogen,

a C₁-C₃ alkoxy (preferably a methoxy),

a group according to the formula —Y—[W]n-Z,

wherein

Y is a radical selected from O, N and C (preferably O);

W is a radical selected from a C₁-C₂ alkyl,

n is equal to 1;

Z represents a phenyl, a pyrrole or a naphthalene, aryl, an imidazole, naphthyl, or a phenanthrene (preferably a phenyl) optionally substituted by at least one radical selected from a C₁-C₃ alkyl, a C₁-C₃ alkoxy and a halogen.

In a particular embodiment, the compound is selected from 4-(benzyloxy)-N-(5-{imidazo[2,1-b][1,3,4]thiadiazol-2-yl}-2-methoxyphenyl)benzamide, N-[5-(1H-1,3-benzodiazol-2-yl)-2-methylphenyl]-4-[(4-chlorophenyl)methoxy]-3-methoxybenzamide, N-[4-(1H-1,3-benzodiazol-2-yl)phenyl]-3-(benzyloxy)benzamide and N-[3-(1H-1,3-benzodiazol-2-yl)phenyl]-4-[(4-chlorophenyl)methoxy]-3-methoxybenzamide, or salts thereof.

An object of the invention is also a compound according to formula (I) or a salt thereof for its use in a method for lifting the latency of HIV in latent HIV-infected cells or for reactivating latent HIV in latent HIV-infected cells in a subject with HIV.

An object of the invention is also a compound according to formula (I) capable of activating the Tat protein, or a salt thereof, for its use in a method for lifting the latency of HIV in latent HIV-infected cells or for reactivating latent HIV in latent HIV-infected cells in a subject with HIV.

An object of the invention is also a compound according to formula (I) having a strong affinity for the Tat-TAR transcription complex, and promoting the transcriptional activity of this protein, or a salt thereof, for its use in a method for lifting HIV latency in latent HIV-infected cells or for reactivating latent HIV in latent HIV-infected cells in an HIV-infected subject.

An object of the invention is also the use of a compound according to formula (I) capable of activating the Tat protein, or a salt thereof, for the preparation of a medication for lifting the latency of HIV in latent HIV-infected cells or for the preparation of a medication for the reactivation of latent HIV in latent HIV-infected cells in an HIV-infected subject.

An object of the invention is also the use of a compound according to formula (I) having a strong affinity for the Tat-TAR transcription complex, and promoting the transcriptional activity of this protein, or a salt thereof, for the preparation of a medication for lifting HIV latency in latent HIV-infected cells or for the preparation of a medication for the reactivation of latent HIV in latent HIV-infected cells in an HIV-infected subject.

An object of the invention is also a method for treating an HIV infection in a subject with HIV, comprising a step according to which one or more compounds according to formula (I) are administered to said patient so as to reactivate the HIV in latent HIV-infected cells of said patient.

An object of the invention is also a method for treating an HIV infection in a subject with HIV, comprising a step according to which one or more compounds according to formula (I) and capable of activating the Tat protein, are administered to said patient so as to reactivate HIV in latent HIV-infected cells of said patient.

An object of the invention is also a method for treating an HIV infection in a subject with HIV, comprising a step according to which one or more compounds according to formula (I) and having a strong affinity for the transcription complex Tat-TAR, and promoting the transcriptional activity of this protein, are administered to said patient so as to reactivate HIV in latent HIV-infected cells of said patient.

Advantageously, such treatment methods also comprise the administration of an antiviral treatment, in particular an antiretroviral treatment, in order in particular to eliminate the latent cells which have been reactivated. The administration to said subject of one or more antiviral agents can be done prior to, simultaneously and/or sequentially to the administration of one or more compounds according to the invention.

According to the invention, the compound is administered parenterally, enterally or cutaneously, preferably parenterally, in particular by subcutaneous or intravenous injection, or enterally, in particular orally.

An object of the invention is also a pharmaceutical composition comprising at least one compound of formula (I) and a pharmaceutically acceptable carrier.

An object of the invention is also a method for treating an HIV-infected patient, said method comprising the steps according to which an effective amount of the pharmaceutical composition comprising at least one compound of formula (I) and a pharmaceutically acceptable carrier is administered to said patient, and an effective amount of one or more antiviral agents is administered to said patient. Advantageously, the compound of formula (I) of said composition is advantageously capable of activating the Tat protein and/or has a strong affinity for the Tat-TAR transcription complex, and promotes the transcriptional activity of this protein.

An object of the invention is also a kit comprising at least one compound according to the invention and at least one antiviral agent and/or at least one other latency lifting agent (LRA), different from the compounds according to the invention. Advantageously, the additional LRAs target a protein other than the Tat protein. In particular, the additional LRAs can target a cellular protein. Advantageously, said compound is conditioned to be used at a concentration comprised between 0.03 μM and 15 μM, in particular a concentration comprised between 3 μM and 10 μM.

An object of the invention is also an in vitro/ex vivo use of a compound according to the invention, for the reactivation of HIV in latent HIV-infected cells.

### BRIEF DESCRIPTION OF THE FIGURES

FIG. 1A Delay gel showing the migration of TAR RNA which is free or complexed with the Tat protein (Tat/TAR) in the presence of different concentrations (0 to 120 nM) of the compound (2) (C-2). Tat (400 nM) was incubated with 200 nM of TAR-FAM (WT or Abulge) for 1.5 h in the absence or presence of the compound (2), before separation on a 0.5×TBE 8% acrylamide gel, and acquisition of the fluorescence image. The proteins from the gel were then transferred to a nitrocellulose membrane for Tat visualization by Western blot.

FIG. 1B Quantification of the affinity of the compound (2) (C-2) for the Tat-TAR transcription complex. The Tat-TAR fluorescence band of duplicate gels was quantified and the results are shown in the graph (AU, arbitrary units). n=2. Means+/−SEM. *, p<0.05; ***, p<0.001 compared to the control without the compound (2) (One-way ANOVA).

FIG. 2 Evaluation of the transactivating activity of the Tat protein of HeLa cells in the presence of 5 μM of the compound (2) (C-2) or of the compound (3) (C-3). HeLa cells were transfected with plasmids containing LTR-firefly and TK-renilla. In the Tat intra condition, Tat is co-transfected. 18 h after transfection the compounds (2) or (3) were added (5 μM), as well as 200 nM Tat for the Tat extra condition. The cells are lysed for the luciferase tests 24 hours later. The firefly/renilla ratio obtained under control conditions was set at 100. n=2. Means+/−SEM.**, p<0.01, ***, p<0.001 and ****, p<0.0001 compared to the drug-free control (One-way ANOVA).

FIG. 3A LRA activity of the compounds (1), (2) (3) (C-1, C-2, C-3) and SAHA (histone deacetylase inhibitor) at 5 μM on J-Lat 9.2 latent cells (derived from Jurkats). J-Lat 9.2 have a complete (single) HIV genome with a non-functional Env gene, and a GFP instead of Nef (Symons, J., and al., Retrovirology, 2017. 14(1): p. 2; Jordan, A., and al., The EMBO journal, 2003. 22(8): p. 1868-77). The activation of these viruses can be followed by the production of GFP and therefore by the detection of green fluorescence in the cells, followed by FACS. n=3. Means+/−SEM.**, p<0.01, compared to the drug-free control (One-way ANOVA).

FIG. 3B LRA activity of the compound (2) (C-2) and SAHA at 5 μM on latent OM10.1 cells (promyeloblasts). These latent cells, after induction, produce infectious viruses which are assayed in the supernatant by ELISA p24. n=2. Means+/−SEM.*, p<0.05 and ***, p<0.001 compared to the control medium alone (One-way ANOVA).

FIG. 4 Evaluation of the specificity of the compound (2) (C-2) for the Tat-TAR complex: the production of HIV-1 in the latent cell line ACH-2 which has a mutated TAR which does not allow transactivation by Tat was measured, after contacting ACH-2 cells with the compound (2) (5 μM), SAHA (5 μM) or bryostatin-1 (BST-1; protein kinase C activator; 10 nM). n=2. Means+/−SEM. ****, p<0.0001 compared to the solvent control (One-way ANOVA).

FIG. 5 Ex vivo LRA activity on quiescent T-CD4⁺ lymphocyte cells of HIV-1 patients. T-CD4 cells were isolated by negative selection before elimination of activated cells (CD25⁺, CD69⁺, HLA-DR⁺) to obtain quiescent cells which were treated with drugs for 18-20 h to avoid reinfections. The LRA activity of the compounds (2) (C-2) and (3) (C-3) was determined by monitoring the viral protein p24 in the supernatant using a commercial ELISA test improved by the use of a luminescent substrate. The detection limit of p24 was 0.2 pg/ml. The response to BST-1 (10 nM) was set at 100% to allow comparison of LRA efficacy between patients (1 point=1 patient).

FIG. 6 Transactivation test on primary T-CD4 cells. Primary T-CD4 cells were isolated from the blood of healthy donors, then they were transfected with plasmids containing Tat, LTR-firefly and TK-renilla. 18 h after transfection different concentrations of the compound (2) (C-2) were added, and the lysed cells for the luciferase tests 24 h later. n=3. Means+/−SEM. *, p<0.05 and ***, p<0.001 compared to the control without C-2 (One-way ANOVA).

### DETAILED DESCRIPTION

### Definitions

“Halogen” means fluorine, chlorine, bromine or iodine.

“Alkyl” means a saturated, linear or branched aliphatic hydrocarbon group. A “C₁-C₃ alkyl” has 1 to 3 carbon atoms. Examples of alkyl (or C₁-C₃ alkyl) are methyl, ethyl, or propyl.

“Heteroaryl” means a mono- or polycyclic (mainly bi-cyclic) group containing conjugated double bonds, each cycle of which contains from 3 to 6 members and of which at least one member contains a heteroatom, in particular a benzimidazole group, an imidazo[2,1-b]-1,3,4-thiadiazole, a benzothiazole, a benzoxazolone, a purine Oxazolone, benzodiazole, quinazoline, quinoxaline, pyridine.

“Alkoxy” or “alkyloxy”, means an alkyl as defined above, attached to the rest of the molecule via an —O— bond (“−0-alkyl”). An example of an alkoxy is in particular a methoxy or ethoxy group.

“Alkylene” means a divalent group, saturated acyclic hydrocarbon, linear or branched. A “C₁-C₆ alkylene” is an alkylene having 1 to 6 carbon atoms, in particular, a methylene, an ethylene, a propylene, a butylene, a pentylene or a hexylene.

“Alkenyl” means an unsaturated, linear or branched acyclic hydrocarbon group having at least one carbon-carbon double bond. A “C₂-C₆ alkenyl” is an alkenyl having 2 to 6 carbon atoms, in particular ethenyl, propenyl, butenyl, pentenyl, or hexenyl.

The expression “substituted by at least” means that the radical is substituted by one or more groups from the list.

In the context of the invention, the terms “subject”, “individual” or “patient” are interchangeable and refer to an animal, preferably to a mammal, even more preferably to a human, including an adult, a child and a newborn.

The terms “treatment” and “treat” refer to a method for alleviating or reducing a disease and/or accompanying symptoms.

In the context of the invention, the term “therapeutic effect” refers to an effect induced by an active principle, or a pharmaceutical composition according to the invention, capable of preventing or delaying the appearance of a disease, or of curing or lessening the effects of a disease.

Compounds

By working on the possibility of targeting viral proteins to reactivate latent cells in the context of HIV treatment, the inventors have identified compounds capable of binding to the Tat-TAR complex and of stabilizing the Tat protein in a conformational state allowing to promote interaction with its target RNA. The compounds according to the invention seem to act by binding to the central region of the structure, namely the main groove around the tryptophan residue, and thus locking the complex into an overactive conformation.

The compounds according to the invention correspond to the formula (I),

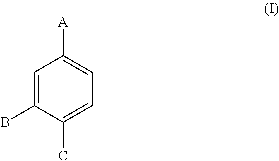

A represents a heteroaryl optionally substituted by at least one halogen.

Preferably, A is selected from a benzimidazole, an imidazo[2,1-b]-1,3,4-thiadiazole, a benzodiazole, a benzothiazole and a benzoxazolone, optionally substituted by at least one halogen, preferentially a chlorine and/or a bromine. For example, A is an optionally chlorine-substituted benzimidazole.

B and C represent, independently of each other, a radical selected from a hydrogen, a C₁-C₆ alkyl, a C₁-C₆ alkoxy, and a group according to formula (II),

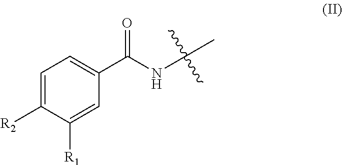

In one embodiment, B is hydrogen, methyl or methoxy and C is a group according to formula (II), wherein R₁ is hydrogen or methoxy and R₂ is the group —Y—[W]n-Z, (or vice versa), wherein Y is O, n=1 and W is methyl and Z is phenyl, optionally substituted by a halogen, preferably chlorine.

Or conversely, C is hydrogen, methyl or methoxy and B is a group according to formula (II), wherein R₁ is hydrogen or methoxy and R₂ is the group —Y—[W]n-Z, (or vice versa), in which Y is O, n=1 and W is methyl and Z is phenyl, optionally substituted by a halogen, preferably chlorine.

In a particular embodiment, A is a benzimidazole or a benzothiazole, C is a methyl or a hydrogen, and B is a group of formula (II) wherein R₁ is methoxy, ethoxy or hydrogen and R₂ a benzyloxy, optionally substituted by a halogen and in particular by a chlorine.

Or conversely, wherein R₁ is benzyloxy, optionally substituted by a halogen and in particular by a chlorine, and R₂ is methoxy, ethoxy or hydrogen.

In another embodiment, A is benzimidazole or benzothiazole, B is methyl or hydrogen, and C is a group of formula (II) wherein R₁ is methoxy, ethoxy or hydrogen and R₂ is benzyloxy, optionally substituted by a halogen and in particular by a chlorine.

Or conversely, wherein R₁ is benzyloxy, optionally substituted by a halogen and in particular by a chlorine, and R₂ is methoxy, ethoxy or hydrogen.

In another embodiment, A is imidazo[2,1-b]-1,3,4-thiadiazole, C is methoxy, and B is a group of formula (II) wherein R₁ is methoxy, ethoxy or hydrogen and R₂ a benzyloxy, optionally substituted by a halogen and in particular by a chlorine.

Or conversely, wherein R₁ is benzyloxy, optionally substituted by a halogen and in particular by a chlorine, and R₂ is methoxy, ethoxy or hydrogen.

In another embodiment, A is imidazo[2,1-b]-1,3,4-thiadiazole, B is methoxy, and C is a group of formula (II) wherein R₁ is methoxy, ethoxy or hydrogen and R₂ a benzyloxy, optionally substituted by a halogen and in particular by a chlorine.

Or conversely, wherein R₁ is benzyloxy, optionally substituted by a halogen and in particular by a chlorine, and R₂ is methoxy, ethoxy or hydrogen.

In one embodiment, Y is a radical selected from O, N and C.

Of course, the person skilled in the art will understand that the valence of the radicals Y is respected.

It is thus understood by the person skilled in the art that the definition “Y is N” is read by the person skilled in the art as being “Y is NH”. Similarly, it is also understood by the person skilled in the art that the definition “Y is C” is read by the person skilled in the art as “C is CH₂”.

In one embodiment, Y is a radical selected from —O—, —NH— and —CH₂₋.

Advantageously, W is a divalent group, that is to say that it is attached by a bond to Y on the one hand and by another bond to Z on the other hand, each bond involving the same carbon or two carbons different from said divalent group.

For example, when W is a divalent “ethyl” group, the group —Y—(W)ₙ—Z includes the two groups shown below:

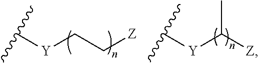

Where Y, Z and n are as defined in this application.

Advantageously, the compound according to formula (I) is selected from compounds (1) to (46) listed in Table 1 below. More advantageously, the compound according to formula (I) is selected from the compounds listed in Table 1, capable of activating the Tat protein and/or which have a strong affinity for the Tat-TAR transcription complex, and promote the transcriptional activity of this protein.

TABLE 1

| | | |
|---|---|---|
| (1) | 4-(benzyloxy)-N-(5-{imidazo[2,1- b][1,3,4]thiadiazol-2-yl}-2- methoxyphenyl)benzamide | 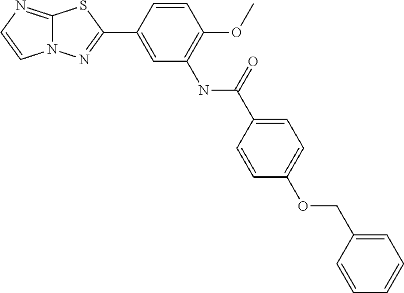 [SMILES: COc1ccc(-c2nn3ccnc3s2)cc1NC(=O)c1ccc(OCc2ccccc2)cc1] |
| (2) | N-[5-(1H-1,3-benzodiazol-2-yl)-2- methylphenyl]-4-[(4- chlorophenyl)methoxy]-3- methoxybenzamide | 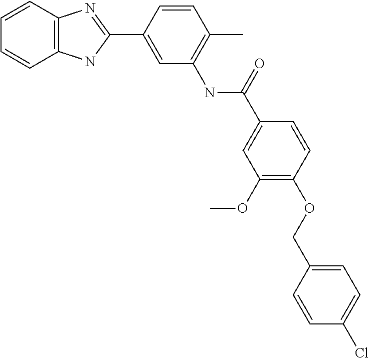 [SMILES: COc1cc(C(=O)Nc2cc(-c3nc4ccccc4[nH]3)ccc2C)ccc1OCc1ccc(Cl)cc1] |
| (3) | N-[4-(1H-1,3-benzodiazol-2- yl)phenyl]-3-(benzyloxy)benzamide | 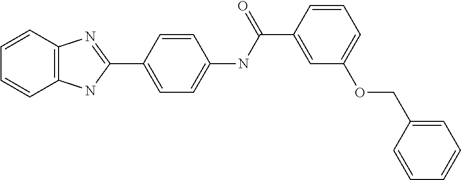 [SMILES: O=C(Nc1ccc(-c2nc3ccccc3[nH]2)cc1)c1cccc(OCc2ccccc2)c1] |
| (4) | N-[3-(1H-1,3-benzodiazol-2- yl)phenyl]-4-[(4- chlorophenyl)methoxy]-3- methoxybenzamide | 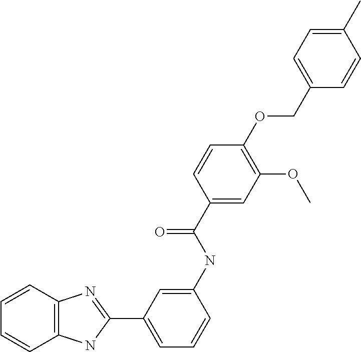 [SMILES: COc1cc(C(=O)Nc2cccc(-c3nc4ccccc4[nH]3)c2)ccc1OCc1ccc(C)cc1] |
| (5) | N-[4-(1H-1,3-benzodiazol-2- yl)phenyl]-3-[(4- chlorophenyl)methoxy]benzamide | 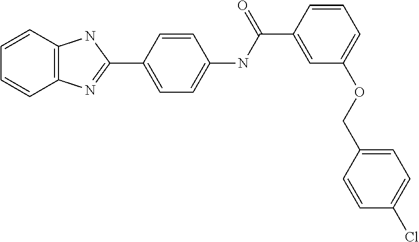 [SMILES: O=C(Nc1ccc(-c2nc3ccccc3[nH]2)cc1)c1cccc(OCc2ccc(Cl)cc2)c1] |
| (6) | N-[4-(1H-1,3-benzodiazol-2- yl)phenyl]-3-(2- phenylethoxy)benzamide | 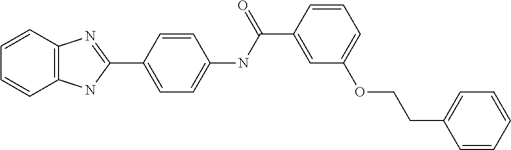 [SMILES: O=C(Nc1ccc(-c2nc3ccccc3[nH]2)cc1)c1cccc(OCCc2ccccc2)c1] |
| (7) | 3-(benzyloxy)-N-[4-(7H-purin-8- yl)phenyl]benzamide | 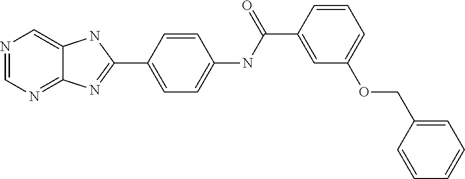 [SMILES: O=C(Nc1ccc(-c2nc3ncncc3[nH]2)cc1)c1cccc(OCc2ccccc2)c1] |
| (8) | 3-(benzyloxy)-N-[4-(5-chloro-1H- 1,3-benzodiazol-2- yl)phenyl]benzamide | 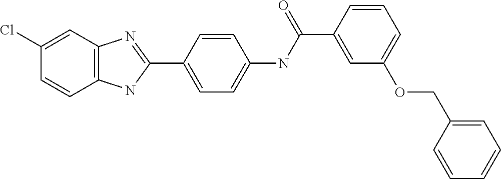 [SMILES: O=C(Nc1ccc(-c2nc3cc(Cl)ccc3[nH]2)cc1)c1cccc(OCc2ccccc2)c1] |
| (9) | N-[4-(1H-1,3-benzodiazol-2- yl)phenyl]-3-[(3,4- dichlorophenyl)methoxy]benzamide | 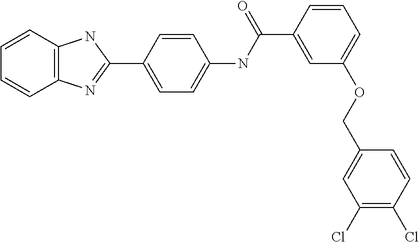 [SMILES: O=C(Nc1ccc(-c2nc3ccccc3[nH]2)cc1)c1cccc(OCc2ccc(Cl)c(Cl)c2)c1] |
| (10) | N-[4-(1H-1,3-benzodiazol-2- yl)phenyl]-3-[(1R)-1- phenylethoxy]benzamide | 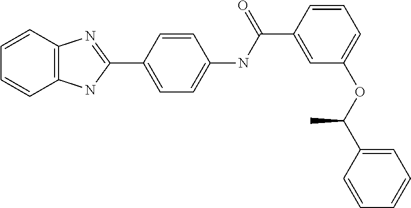 [SMILES: C[C@@H](Oc1cccc(C(=O)Nc2ccc(-c3nc4ccccc4[nH]3)cc2)c1)c1ccccc1] |
| (11) | N-[4-(1H-1,3-benzodiazol-2- yl)phenyl]-3-{[(1E)-2- phenylethenyl]oxy}benzamide | 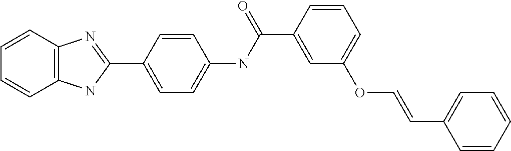 [SMILES: O=C(Nc1ccc(-c2nc3ccccc3[nH]2)cc1)c1cccc(O/C=C/c2ccccc2)c1] |
| (12) | N-[3-(1H-1,3-benzodiazol-2- yl)phenyl]-4-[(4- bromophenyl)methoxy]-3- methoxybenzamide | 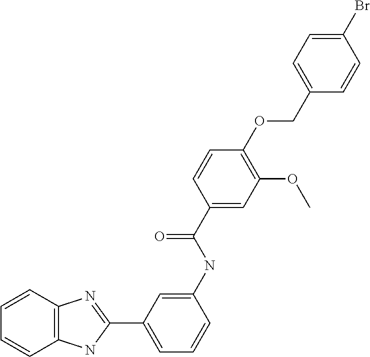 [SMILES: COc1cc(C(=O)Nc2cccc(-c3nc4ccccc4[nH]3)c2)ccc1OCc1ccc(Br)cc1] |
| (13) | N-[3-(1H-1,3-benzodiazol-2- yl)phenyl]-4-(benzyloxy)-3- methoxybenzamide | 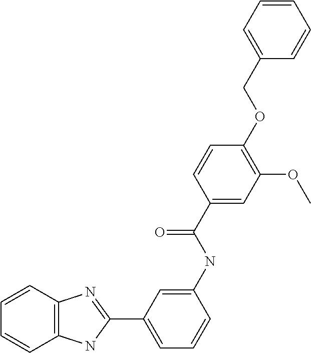 [SMILES: COc1cc(C(=O)Nc2cccc(-c3nc4ccccc4[nH]3)c2)ccc1OCc1ccccc1] |
| (14) | N-[3-(1H-1,3-benzodiazol-2- yl)phenyl]-4-[(4- chlorophenyl)methoxy]-3- ethoxybenzamide | 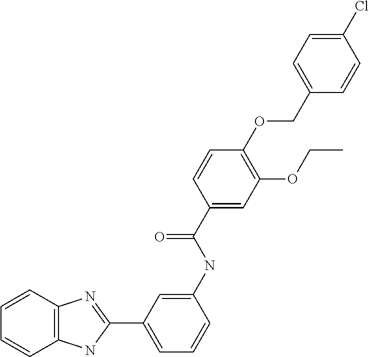 [SMILES: CCOc1cc(C(=O)Nc2cccc(-c3nc4ccccc4[nH]3)c2)ccc1OCc1ccc(Cl)cc1] |
| (15) | N-[3-(1H-1,3-benzodiazol-2- yl)phenyl]-3-methoxy-4-[(1S)-1- phenylethoxy]benzamide | 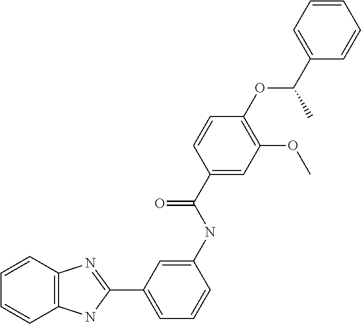 [SMILES: COc1cc(C(=O)Nc2cccc(-c3nc4ccccc4[nH]3)c2)ccc1O[C@@H](C)c1ccccc1] |
| (16) | N-[3-(1H-1,3-benzodiazol-2- yl)phenyl]-3-(benzyloxy)-4-[(4- chlorophenyl)methoxy]benzamide | 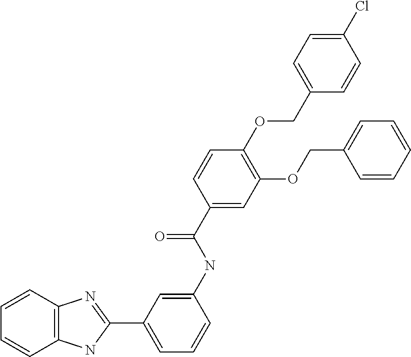 [SMILES: O=C(Nc1cccc(-c2nc3ccccc3[nH]2)c1)c1ccc(OCc2ccc(Cl)cc2)c(OCc2ccccc2)c1] |
| (17) | N-[3-(1,3-benzothiazol-2- yl)phenyl]-4-[(4- chlorophenyl)methoxy]-3- methoxybenzamide | 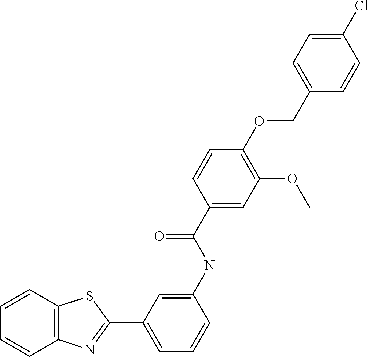 [SMILES: COc1cc(C(=O)Nc2cccc(-c3nc4ccccc4s3)c2)ccc1OCc1ccc(Cl)cc1] |
| (18) | N-[3-(1H-1,3-benzodiazol-2- yl)phenyl]-4-(benzylsulfanyl)-3- methoxybenzamide | 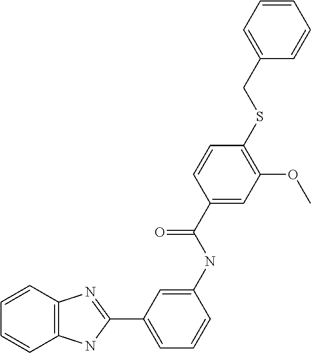 [SMILES: COc1cc(C(=O)Nc2cccc(-c3nc4ccccc4[nH]3)c2)ccc1SCc1ccccc1] |
| (19) | 4-(benzyloxy)-N-(3-{imidazo[2,1- b][1,3,4]thiadiazol-2-yl}phenyl)-3- methoxybenzamide | 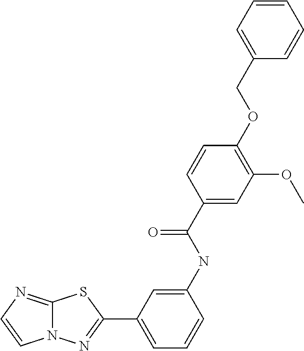 [SMILES: COc1cc(C(=O)Nc2cccc(-c3nn4ccnc4s3)c2)ccc1OCc1ccccc1] |
| (20) | 4-[(4-chlorophenyl)methoxy]-N-(3- {imidazo[2,1-b][1,3,4]thiadiazol-2- yl}phenyl)-3-methoxybenzamide | 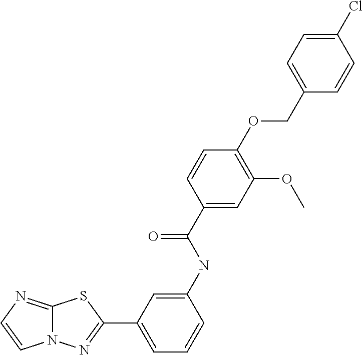 [SMILES: COc1cc(C(=O)Nc2cccc(-c3nn4ccnc4s3)c2)ccc1OCc1ccc(Cl)cc1] |
| (21) | N-(3-{imidazo[2,1- b][1,3,4]thiadiazol-2-yl}phenyl)-4- [(1S)-1-phenylethoxy]benzamide | 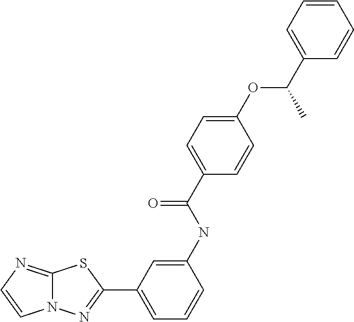 [SMILES: C[C@H](Oc1ccc(C(=O)Nc2cccc(-c3nn4ccnc4s3)c2)cc1)c1ccccc1] |
| (22) | 4-(benzyloxy)-3-ethoxy-N-(3- {imidazo[2,1-b][1,3,4]thiadiazol-2- yl}phenyl)benzamide | 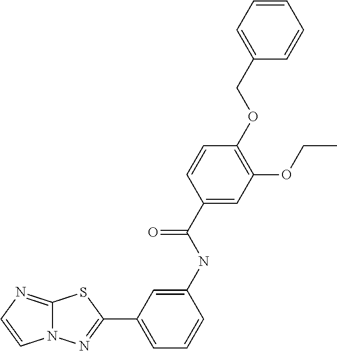 [SMILES: CCOc1cc(C(=O)Nc2cccc(-c3nn4ccnc4s3)c2)ccc1OCc1ccccc1] |
| (23) | 4-[(4-fluorophenyl)methoxy]-N-(3- {imidazo[2,1-b][1,3,4]thiadiazol-2- yl}phenyl)-3-methoxybenzamide | 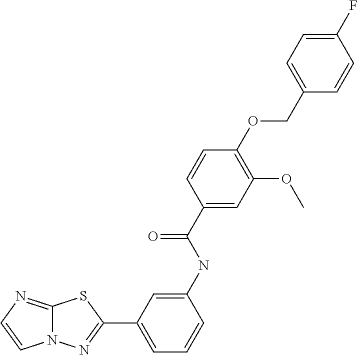 [SMILES: COc1cc(C(=O)Nc2cccc(-c3nn4ccnc4s3)c2)ccc1OCc1ccc(F)cc1] |
| (24) | 4-(benzyloxy)-N-(3- {[1,3]oxazolo[4,5-b]pyridin-2- yl}phenyl)benzamide | 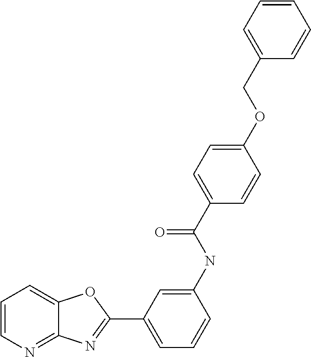 [SMILES: O=C(Nc1cccc(-c2nc3ncccc3o2)c1)c1ccc(OCc2ccccc2)cc1] |
| (25) | N-[5-(1,3-benzothiazol-2-yl)-2- methylphenyl]-4- (benzyloxy)benzamide | 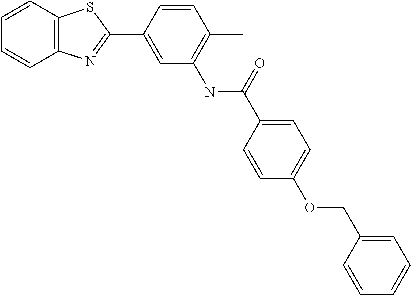 [SMILES: Cc1ccc(-c2nc3ccccc3s2)cc1NC(=O)c1ccc(OCc2ccccc2)cc1] |
| (26) | N-[3-(1H-1,3-benzodiazol-2- yl)phenyl]-3-butoxybenzamide | 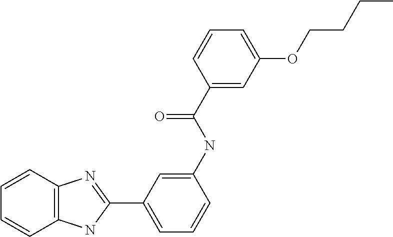 [SMILES: CCCCOc1cccc(C(=O)Nc2cccc(-c3nc4ccccc4[nH]3)c2)c1] |
| (27) | N-[3-(1H-1,3-benzodiazol-2- yl)phenyl]-3-phenoxybenzamide | 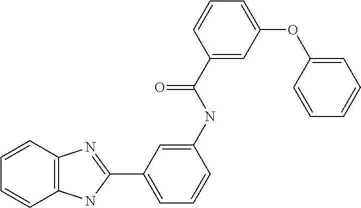 [SMILES: O=C(Nc1cccc(-c2nc3ccccc3[nH]2)c1)c1cccc(Oc2ccccc2)c1] |
| (28) | N-[3-(1H-1,3-benzodiazol-2- yl)phenyl]-3-methoxy-4-[2- (morpholin-4-yl)-2- oxoethoxy]benzamide | 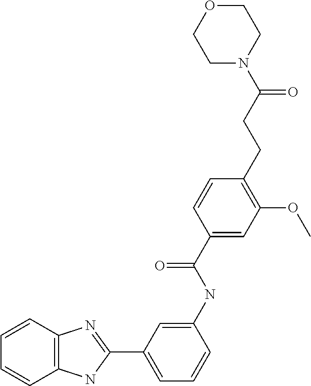 [SMILES: COc1cc(C(=O)Nc2cccc(-c3nc4ccccc4[nH]3)c2)ccc1CCC(=O)N1CCOCC1] |
| (29) | N-[5-(1,3-benzoxazol-2-yl)-2- methoxyphenyl]-4- (benzyloxy)benzamide | 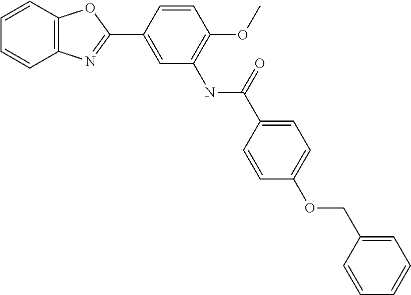 [SMILES: COc1ccc(-c2nc3ccccc3o2)cc1NC(=O)c1ccc(OCc2ccccc2)cc1] |
| (30) | N-[5-(1H-1,3-benzodiazol-2-yl)-2- methylphenyl]-4- (benzyloxy)benzamide | 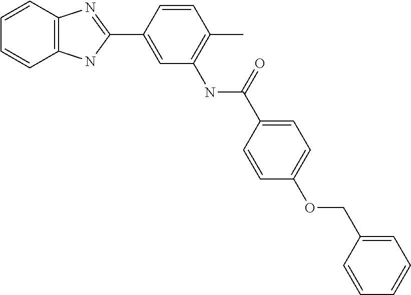 [SMILES: Cc1ccc(-c2nc3ccccc3[nH]2)cc1NC(=O)c1ccc(OCc2ccccc2)cc1] |
| (31) | N-[3-(1H-1,3-benzodiazol-2-yl)-4- chlorophenyl]-3,4,5- triethoxybenzamide | 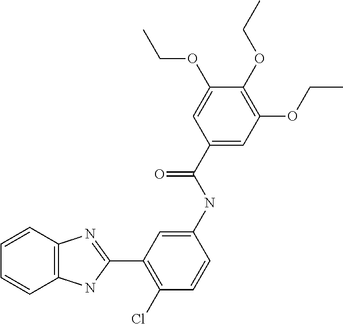 [SMILES: CCOc1cc(C(=O)Nc2ccc(Cl)c(-c3nc4ccccc4[nH]3)c2)cc(OCC)c1OCC] |
| (32) | 3-(benzyloxy)-N-(2-methyl-5-{3- methyl-[1,2,4]triazolo[3,4- b][1,3,4]thiadiazol-6- yl}phenyl)benzamide | 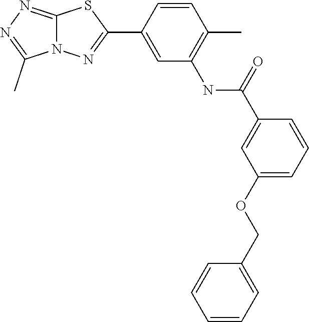 [SMILES: Cc1ccc(-c2nn3c(C)nnc3s2)cc1NC(=O)c1cccc(OCc2ccccc2)c1] |
| (33) | N-[5-(1,3-benzoxazol-2-yl)-2- methoxyphenyl]-3-[(4- fluorophenyl)methoxy]benzamide | 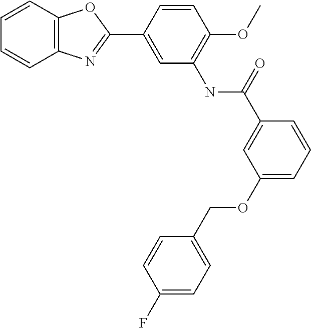 [SMILES: COc1ccc(-c2nc3ccccc3o2)cc1NC(=O)c1cccc(OCc2ccc(F)cc2)c1] |
| (34) | N-[5-(1,3-benzoxazol-2-yl)-2- methoxyphenyl]-4-[(1R)-1- phenylethoxy]benzamide | 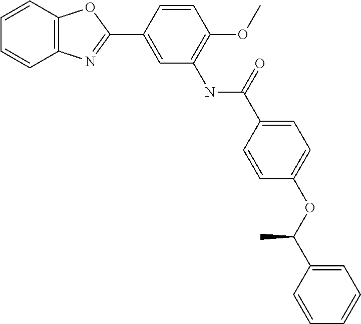 [SMILES: COc1ccc(-c2nc3ccccc3o2)cc1NC(=O)c1ccc(O[C@H](C)c2ccccc2)cc1] |
| (35) | N-[5-(1,3-benzoxazol-2-yl)-2- methoxyphenyl]-4-(benzyloxy)-3,5- difluorobenzamide | 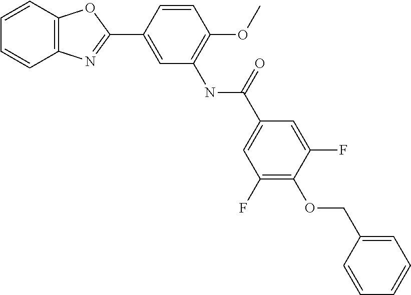 [SMILES: COc1ccc(-c2nc3ccccc3o2)cc1NC(=O)c1cc(F)c(OCc2ccccc2)c(F)c1] |
| (36) | N-[5-(1,3-benzoxazol-2-yl)-2- methoxyphenyl]-3- (benzyloxy)benzamide | 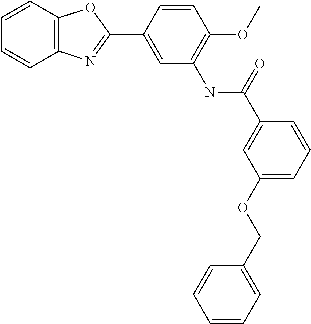 [SMILES: COc1ccc(-c2nc3ccccc3o2)cc1NC(=O)c1cccc(OCc2ccccc2)c1] |
| (37) | 4-(benzyloxy)-N-[2-methoxy-5- (pyridin-3-yl)phenyl]benzamide | 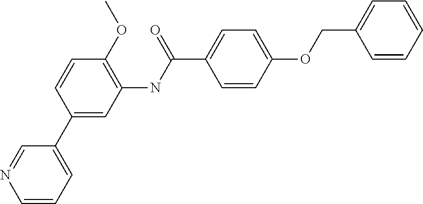 [SMILES: COc1ccc(-c2cccnc2)cc1NC(=O)c1ccc(OCc2ccccc2)cc1] |
| (38) | 4-(benzyloxy)-N-[2-methoxy-5- (quinazolin-6-yl)phenyl]benzamide | 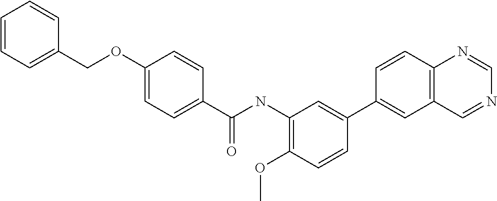 [SMILES: COc1ccc(-c2ccc3ncncc3c2)cc1NC(=O)c1ccc(OCc2ccccc2)cc1] |
| (39) | 4-(benzyloxy)-N-[2-methoxy-5- (quinoxalin-6-yl)phenyl]benzamide | 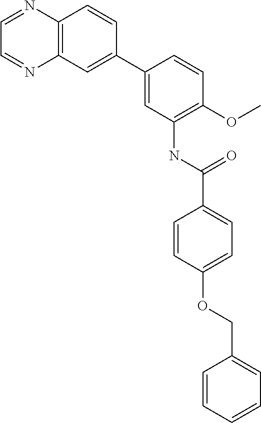 [SMILES: COc1ccc(-c2ccc3nccnc3c2)cc1NC(=O)c1ccc(OCc2ccccc2)cc1] |
| (40) | 4-(benzyloxy)-N-(5-{imidazo[1,2- a]pyrimidin-6-yl}-2- methoxyphenyl)benzamide | 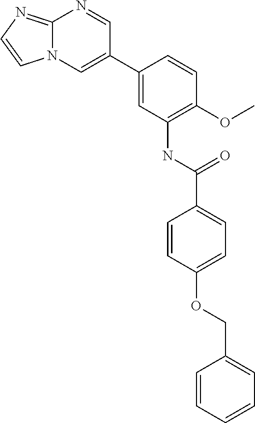 [SMILES: COc1ccc(-c2cnc3nccn3c2)cc1NC(=O)c1ccc(OCc2ccccc2)cc1] |
| (41) | 4-(benzyloxy)-N-(2-methoxy-5- {[1,3]thiazolo[5,4-b]pyridin-2- yl}phenyl)benzamide | 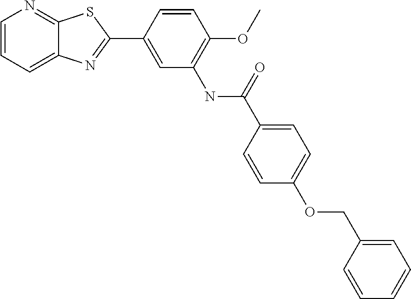 [SMILES: COc1ccc(-c2nc3cccnc3s2)cc1NC(=O)c1ccc(OCc2ccccc2)cc1] |
| (42) | N-[5-(1,3-benzoxazol-2-yl)-2- methoxyphenyl]-4- (benzyloxy)benzamide | 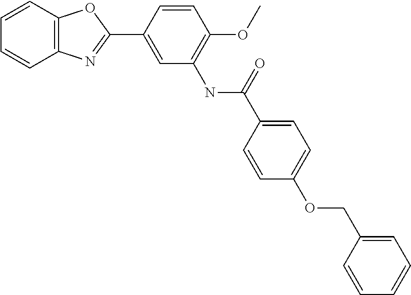 [SMILES: COc1ccc(-c2nc3ccccc3o2)cc1NC(=O)c1ccc(OCc2ccccc2)cc1] |
| (43) | N-[5-(1,3-benzoxazol-2-yl)-2- methylphenyl]-4- (benzyloxy)benzamide | 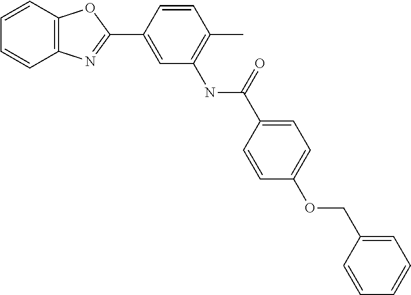 [SMILES: Cc1ccc(-c2nc3ccccc3o2)cc1NC(=O)c1ccc(OCc2ccccc2)cc1] |
| (44) | N-[5-(1,3-benzoxazol-2-yl)-2- methoxyphenyl]-4- (benzyloxy)benzene-1- carbothioamide | 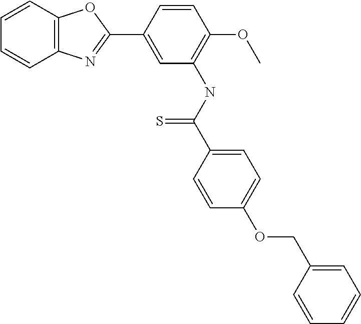 [SMILES: COc1ccc(-c2nc3ccccc3o2)cc1NC(=S)c1ccc(OCc2ccccc2)cc1] |
| (45) | 4-benzamido-N-[5-(1,3- benzoxazol-2-yl)-2- methoxyphenyl]benzamide | 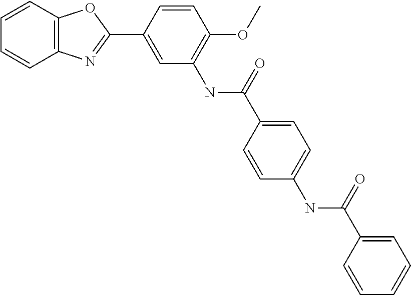 [SMILES: COc1ccc(-c2nc3ccccc3o2)cc1NC(=O)c1ccc(NC(=O)c2ccccc2)cc1] |
| (46) | 2-{3-[(1E)-2-[4- (benzyloxy)phenyl]diazen-1-yl]-4- methoxyphenyl}-1,3-benzoxazole | 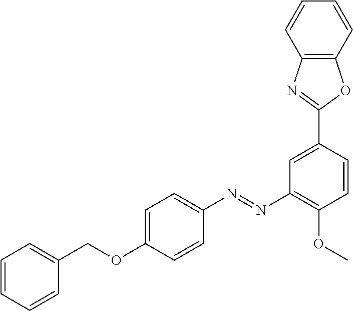 [SMILES: COc1ccc(-c2nc3ccccc3o2)cc1/N=N/c1ccc(OCc2ccccc2)cc1] |

In a particular embodiment, the compound according to formula (I) is selected from compounds (1) to (9), (11) to (17), (20) to (23), (25), (29), (30), (33), (34), (36) to (38), (40) to (43) listed in Table 1.

In one embodiment, the compound corresponds to formula (I) on the condition that it is not selected from compounds (10), (18), (19), (24), (26), (27), (31), (32), (39) and (45) listed in Table 1.

In one embodiment, the compound corresponds to formula (I) on the condition that it is not selected from compounds (10), (18), (19), (24), (27), (32) and (39).

In one embodiment, the compound is selected from the compounds corresponding to formula (I) listed in Table 1, on the condition that it is capable of activating the Tat protein.

In one embodiment, the compound is selected from the compounds corresponding to formula (I) listed in Table 1, on the condition that it has a high affinity for the Tat-TAR transcription complex, and promotes the activity transcription of this protein.

The invention also relates to a pharmaceutically acceptable salt of a compound of formula (I).

It should also be understood that certain compounds of formula (I) can exist in solvated as well as unsolvated forms such as, for example, hydrated forms. It should be understood that the invention encompasses all those solvated forms which are capable of binding to the Tat-TAR complex.

Pharmaceutical Composition

An object of the present invention is also a pharmaceutical composition comprising one or more compounds of formula (I) as active principle and a pharmaceutically acceptable carrier. Advantageously, the compound(s) of the composition are selected from the compounds of formula (I) capable of activating the Tat protein and/or which have a strong affinity for the Tat-TAR transcription complex, and promote the transcriptional activity of this protein.

In the context of the invention, the terms “active principle”, “active ingredient” and “active pharmaceutical ingredient” are equivalent and refer to a component of a pharmaceutical composition having a therapeutic effect.

In the context of the invention, the expression “excipient” or “pharmaceutically acceptable carrier” refers to any ingredient, except the active ingredients, which is present in a pharmaceutical composition. Its addition may aim at imparting a particular consistency or other physical or taste properties to the final product. A pharmaceutically acceptable excipient or carrier must be devoid of any interaction, in particular chemical interaction, with the active principles.

The pharmaceutical composition according to the invention can be formulated in a form intended for topical, enteral, oral, parenteral, intranasal, intravenous, intra-arterial, intramuscular, subcutaneous or intraocular administration and the like. In particular, the pharmaceutical composition according to the invention can be formulated for topical, enteral, oral, parenteral, intranasal, intravenous, intra-arterial, intramuscular, subcutaneous or intraocular administration and the like.

For oral administration, the composition can be formulated in conventional oral dosage forms such as tablets, capsules, powders, granules and liquid preparations such as syrups, elixirs and concentrated drops. Use can be made of non-toxic solid carriers or diluents, which comprise, for example, pharmaceutical grades of mannitol, lactose, starch, magnesium stearate, sodium saccharin, talc, cellulose, glucose, sucrose, magnesium, carbonate and the like. For tablets, binders, which are agents imparting cohesive qualities to powdered materials, can also be used. For example, starch, gelatin, sugars such as lactose or dextrose, and natural or synthetic gums can be used as binders. Disintegrants can also be used in tablets to facilitate tablet bursting. Disintegrants comprise starches, clays, celluloses, algins, gums and cross-linked polymers.

For transdermal administration, the composition can be formulated as an ointment, cream or gel and suitable penetrants or detergents can be used to facilitate permeation, such as dimethylsulfoxide, dimethylacetamide and dimethylformamide.

For transmucosal administration, nasal sprays, rectal or vaginal suppositories can be used. The active compound can be incorporated into any of the known suppository bases by processes known in the art. Examples of such bases comprise cocoa butter, polyethylene glycols (carbowaxes), polyethylene sorbitan monostearate, and mixtures thereof with other compatible materials to alter the melting point or dissolution rate.

The pharmaceutical compositions according to the invention may be formulated to release the active medication substantially immediately after administration or at any predetermined time or period of time after administration.

Treatment Method and Dosage

The compounds according to the present invention are agents for lifting latency capable of reactivating HIV in latent HIV-infected cells.

The invention therefore proposes a method for treating HIV according to which one or more compounds according to formula (I), or a composition according to the invention are administered to a subject with HIV and having latent infected cells. Advantageously, the treatment method also comprises administering an antiviral agent.

The invention more particularly provides a method for treating HIV latency in HIV-infected cells in a subject with HIV. This latency treatment method can advantageously be integrated into a more general method for treating HIV according to which one or more antiviral agents are also administered to said subject. Since the purpose of the compounds according to the invention is to reactivate HIV in latent HIV-infected cells, it will most often be necessary to supplement the treatment with the compounds according to the invention with treatment with antiviral agents in order to neutralize the production of HIV which will have been reactivated. In particular, the subject can be treated with antiviral agents at the time of the latency treatment by means of one or more compounds according to the invention; and/or have been treated with antiviral agents prior to the treatment of latency by means of one or more compounds according to the invention; and/or be treated with antiviral agents after latency treatment with one or more compounds according to the invention.

The compounds according to the invention can also be used in vitro/ex vivo to reactivate HIV in latent HIV-infected cells. Such a use can in particular be useful in vitro for treating cells of the reservoir, previously taken from an HIV-infected patient. Once the cells have been treated in vitro, that is to say once the latent cells in the reservoir have been reactivated in vitro, they can be reinjected into the patient.

An object of the present invention is also the compounds and pharmaceutical compositions described above for their use as medications, more particularly for their use in the treatment of an HIV infection, in particular for the treatment of HIV latency in HIV-infected cells; the use of the compounds and pharmaceutical compositions described above for the manufacture of a medication intended for the treatment of an HIV infection, in particular for the treatment of HIV latency in HIV-infected cells; a method for treating HIV infection, in particular for treating HIV latency in HIV infected cells, in an HIV infected patient comprising administering a therapeutically effective amount of the compounds and pharmaceutical compositions described above.

The term “therapeutically effective amount” is understood to mean an amount allowing to reactivate HIV in HIV-infected cells and in which HIV is latent. It is obvious that the amount to be administered can be adapted by the person skilled in the art according to the subject to be treated, the state of progress of the disease, etc. In particular, the doses and the administration schedule may depend on the stage and severity of the disease to be treated, as well as the weight, age and overall health of the subject to be treated, as well as the judgment of the doctor.

In a particular embodiment, the present invention relates to the pharmaceutical or vaccine composition described above for its use in the treatment of an infection by HIV, in particular type I HIV, in combination with an antiviral treatment; the use of the pharmaceutical or vaccine composition described above for the manufacture of a medication or vaccine intended for the treatment of an HIV infection, in particular type I HIV, in combination with an antiviral treatment; and to a method for treating an HIV infection, in particular type I HIV, in an HIV-infected patient comprising the administration of a therapeutically effective amount of the pharmaceutical or vaccine composition described above, and the administration of an antiviral treatment.

The antiviral treatment of HIV infected-patients is well known to the person skilled in the art. In particular, different antiretrovirals belonging to several different medication classes are available.

In particular, the antiretrovirals can be reverse transcriptase inhibitors, preferably selected from Abacavir, emtricitabine, lamivudine, tenofovir disoproxil fumarate, zidovudine, efavirenz, etravirine, nevirapine, rilpivirine, and even doravirine; protease inhibitors preferentially selected from Atazanavir, darunavir, fosamprenavir, ritonavir, saquinavir, tipranavir, lopinavir; fusion inhibitors, such as enfuvirtide, CCR5 antagonists, such as maraviroc, integrase inhibitors such as dolutegravir, raltegravir, elvitegravir, and even cabotegravir or bictegravir, post-attachment inhibitors, in particular ibalizumab, combinations, in particular dolutegravir, abacavir and lamivudine, or zidovudine, abacavir and lamivudine, or efavirenz, emtricitabine and tenofovir, or efavirenz, lamivudine and tenofovir, or bictefravir, emtricitabine and tenofovir, or elvitegravir, cobicistat, emtricitabine and tenofovir, or rilofopivirine, emtricitabine and tenofovir, or abacavir and lamivudine, or abacavir, lamivudine and zidovudine, or emtricitabine and tenofovir, or lamivudine and zidovudine, or lopinavir and ritonavir, or atazanavir and cobicistat, or darunavir and cobicistat, or darunavir, cobicistat, emtricitabine and tenofovir, or dolutegravir and rilpivirine, or Lamivudine and tenofovir.

Thus, when reference is made here to an antiviral treatment, it is understood in particular to be any antiretroviral medication or combination thereof, as described above.

Preferably, the compound according to the invention or the pharmaceutical composition according to the invention is administered by enteral or parenteral administration. When it is administered parenterally, the compound according to the invention or the pharmaceutical composition according to the invention is preferably administered intravenously. When it is administered enterally, the compound according to the invention or the pharmaceutical composition according to the invention is preferably administered orally.

The compound according to the invention or the pharmaceutical composition according to the invention can be administered in a single dose or in multiple doses.

Preferably, the treatment is administered regularly, preferably between every day and every month, more preferably between every day and every two weeks, more preferably between every day and every week, even more preferably the treatment is administered every day. In a particular embodiment, the treatment is administered several times a day, preferably 2 or 3 times a day, even more preferably 3 times a day.

The duration of treatment with the compound according to the invention or the pharmaceutical composition according to the invention is preferably comprised between 1 day and 20 weeks, more preferably between 5 days and 10 weeks, even more preferably between 5 days and 4 weeks, even more preferably between 5 days and 2 weeks. In a particular embodiment, the duration of the treatment is at least 1 week.

The form of the pharmaceutical compositions, the route of administration and the dose of administration of the compound according to the invention, or of the pharmaceutical composition according to the invention can be adjusted by the person skilled in the art according to the type and severity of HIV infection and the patient, especially their age, weight, sex and general physical condition.

The invention also proposes a kit comprising at least one compound according to the invention and at least one antiviral agent, in particular at least one antiretroviral agent and/or at least one other latency lifting agent (LRA). Advantageously, the compound of the kit is packaged to be used at a concentration comprised between 0.03 μM and 15 μM. In one embodiment, the compound is packaged to be used at a concentration comprised between 3 μM and 15 μM. In another embodiment, the compound is packaged to be used at a concentration comprised between 0.03 μM and 0.1 μM.

### Examples

Evaluation of the Affinity of the Compounds According to the Invention for the Tat Protein and the Tat-TAR Transcription Complex

The recombinant Tat protein (86 residues, isolate BH10) was produced in E. coli and purified on heparin-Sepharose then by HPLC as described (Mol Biol Cell 15 (2004), 2347). The compound (2) (MolPort) being fluorescent (λex 320 nm; λem 410 nm), the fluorescence polarization technique (Methods Appl. Fluoresc. (2016) 4(2)) and a PTI fluorimeter were used for the study of the binding of said compound (17 nM) to the Tat protein (1-60 μM) in citrate buffer (50 mM citrate; 150 mM NaCl; pH 7.2). A Kd of the order of 5 μM was measured. An identical value (˜4 μM) was obtained by surface plasmon resonance (SPR; Biacore) by passing the compound (2) in citrate buffer over immobilized Tat on a CMS chip. These measurements show that the compound (2) has a fairly modest affinity for Tat, and above all this Kd value was well above the concentration sufficient for the LRA effect of the compound (2) ex vivo which is 50-100 nM (see below).

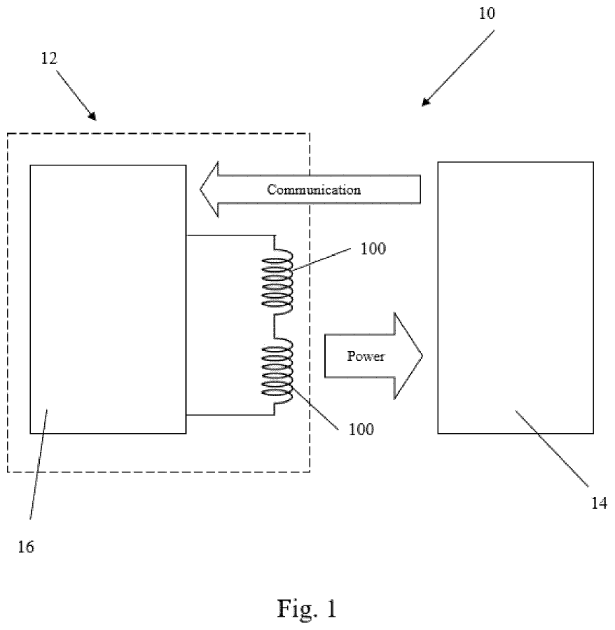

The affinity of the compound (2) for the Tat-TAR complex was studied by retardation gel. For this purpose, target RNA (TAR 30 bases; 200 nM; WT or A bulge, that is to say lacking the outgrowth allowing the binding of Tat) marked with fluorescein (Sigma) was mixed with 400 nM Tat+/−compound (2) in 30 mM Tris (pH 7.5), 100 mM KCl, 2 mM dithiothreitol (DTT), 1 U RNAsin (Promega)/μl, 0.03% NP40, 10% glycerol, 5 μg polyIC (Sigma)/ml, and RNase-free water. After incubation for 1.5 hours at 25° C., the mixture is deposited on an 8% acrylamide gel in tris-borate-EDTA (TBE; migration for 1 hour 20 minutes at 4° C.) (FIG. 1A). The free probe migrates to the front and is delayed if Tat binds thereto. The fluorescence of the gel is first visualized with a Chemidoc (Biorad) to detect fluorescein (that is to say TAR). The proteins of the gel are then transferred onto a nitrocellulose membrane to detect Tat by Western blot using an anti-Tat monoclonal antibody (mouse) (sc-65912) then a rabbit anti-mouse-peroxidase antibody (Jackson Immunoresearch). The membrane is revealed with ECL select (GE Healthcare) visualized with the Chemidoc. The presence of the compound (2) promotes the Tat-TAR interaction. As shown in FIG. 1B, the compound (2) has a very strong affinity for the Tat-TAR transcription complex (˜80 nM).

Evaluation of the Cytotoxicity of the Compounds According to the Invention

The cytotoxicity of the compound (2) and of the other molecules was evaluated on primary human T CD4 cells (cells capable of becoming latent). The cells were purified from healthy donor blood (convention with EFS) by Ficoll-Hypaque gradient then negative selection of T-CD4 (Miltenyi Biotec 130-096-533). They were then activated by phytohaemagglutinin (1 μg/ml) then by interleukin-2 (50 U/ml) for 5-7 days (Nature comms 2018 9(1) 2251). They are then incubated (1 million/ml) for 24 h with the compound (2) (1 nM-100 μM) in 96-well plates. The cell viability is tested using the CellTiter-Blue reagent (Promega) as recommended by the manufacturer before reading OD using a Tecan SPARK 10M. Concentrations >30 μM were mildly cytotoxic. Similar results were obtained using human cell lines HEK (293/T17 from ATCC) or HeLa (CCL2 from ATCC). No significant toxicity was observed for concentrations <10 μM, regardless of the compound.

Evaluation of the Effect of the Compounds According to the Invention on Transactivation by the Tat Protein

The starting hypothesis was that since latent cells express a certain level of viral proteins, if transactivation by Tat is activated, cells latently infected with HIV-1 can be reactivated to then be targeted by ART and/or lysed by cytotoxic T cells.

For this test, the effect of the molecules on transactivation by the extracellular or intracellular Tat protein was monitored by the activity of two luciferases. On the one hand the Firefly luciferase, which is under the control of a viral promoter LTR dependent on the Tat protein, and on the other hand the Renilla luciferase (internal control), under the control of a Thymidine kinase cellular promoter independent of the Tat protein. Transactivation is expressed by the Firefly/Renilla ratio. All plasmids and the procedure have been described (Mol Biol Cell 15 (2004), 2347).

This series of experiments was carried out in the HeLa line. Cells were transfected with PEImax (Nature Comms 9 (2018) 2251). The drugs and Tat (if Tat extra) were added 18 h after transfection as indicated, and left for 24 h before lysing the cells. The luciferases are then assayed using Promega's Dual-Glo kit and a Tecan SPARK 10M plate reader. Extracellular Tat has the property of being able to enter into the cells. This results in a very low intracellular concentration of Tat (Mol Biol Cell 15 (2004), 2347), very probably close to that present in the latent cells.

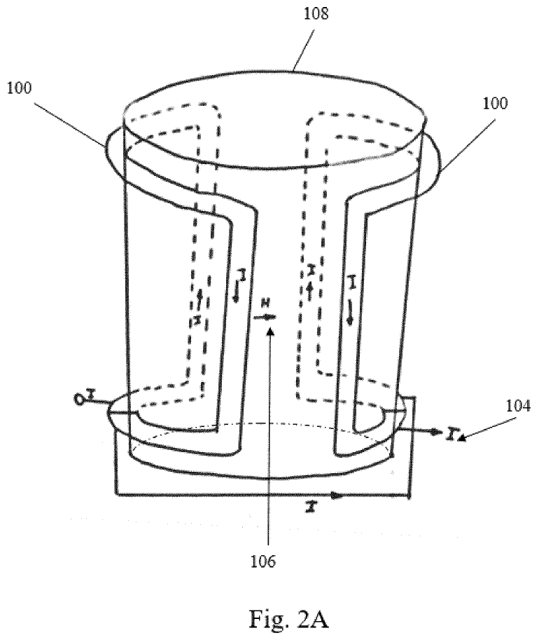

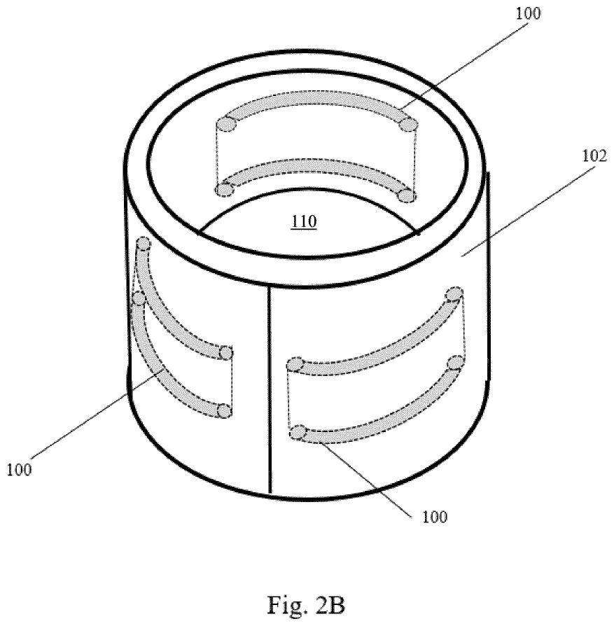

Thus, the recombinant Tat alone added to the cells has no significant effect compared to the control. On the other hand, with compounds (2) and (3) at 5 μM, the transactivation by extracellular Tat is very strong, 60 times (C-2) and 108 times (C-3) that of the control (FIG. 2; Tat extra).

The same effects of compounds (2) and (3) on transactivation by Tat were noted when tested with the intracellular Tat protein. In this case, Tat is transfected, which induces a high concentration of intracellular Tat. In the presence of 5 μM of the compounds, the transactivation by the intracellular Tat protein was multiplied 3.9 times (C-2) and 9 times (C-3), in comparison with the negative control (FIG. 2; Tat intra). The compounds (2) and (3) therefore act directly on the transcriptional activity of Tat and not by promoting its entry into the cell.

Evaluation of the LRA Activity of the Compounds According to the Invention on Latent Cell Lines

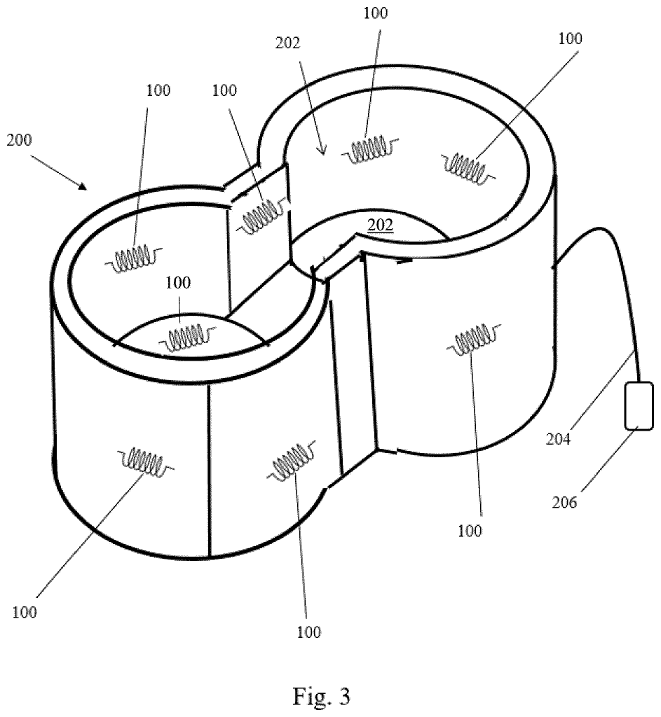

To test the LRA effect of the molecules, JLat9.2 cells were first used. This latent cell line (source: NIH AIDS Reagents Program, NARP) derived from Jurkat cells contains an HIV genome with an inactivated Env gene and a GFP instead of Nef (Retrovirology, 14 (2017) 2). The lifting of latency is accompanied by the emission of fluorescence from GFP. The cells were treated with molecules at 5 μM for 24 h before analysis of the cells with a FACScalibur to determine the % of cells emitting fluorescence. It can be seen in FIG. 3A that the compound (2) at 5 μM has a significant LRA activity, of the order of 20-30% of that of SAHA. Similarly, the compound (3) confirms the results in transactivation with an LRA activity close to that of SAHA. The compound (1) also has significant activity.

The results for the compound (2) were confirmed using another latent cell line, OM10.1 (promyeloblasts; source NARP; PLoS Pathog. 11 (2015) 1) which produces infectious HIV after induction. It was assayed by the viral antigen p24 (HIV-1 capsid protein) in the supernatant using an ELISA kit (Innotest). On this more physiological latency model, the compound (2) shows better activity, of the order of 40% of that of SAHA (FIG. 3B).

Specificity of the Compounds According to the Invention

The specificity of the compounds was evaluated using the latent cell line ACH-2 (Source NARP) which has an HIV genome with a mutated TAR (Retrovirology, 14 (2017) 2) which does not allow transactivation by Tat (J. Virol. 68 (1994) 1993). Once induced, these cells produce infectious virions. For these tests, the viral antigen p24 in the cell supernatant was assayed with the Innotest p24 ELISA kit.

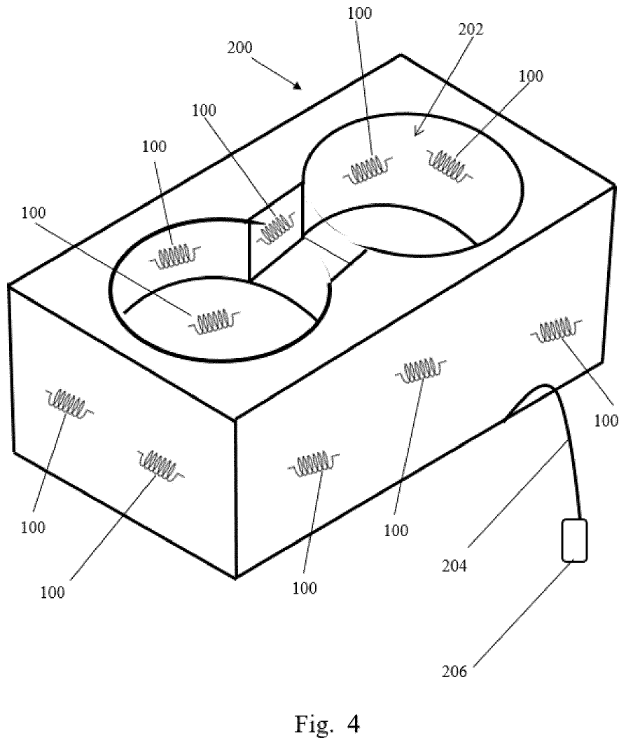

It can be seen (FIG. 4) that the compound (2) (5 ρM) is incapable of inducing the production of HIV-1 in these cells whereas other LRAs (SAHA and BST-1) can. The LRA activity of the compound (2) therefore passes through the Tat-TAR viral transcription complex.

LRA Activity of the Compounds According to the Invention on the Latent Cells of HIV-1 Patients

The ex vivo LRA activity of the compounds according to the invention was tested on latent T-CD4⁺ lymphocyte cells from HIV-1 patients. All 8 patients had been under ART treatment for at least 6 months and their viral load was undetectable. Blood samples (20 ml) were taken (agreement with Montpellier University Hospital, Resp: Dr Edouard Tuaillon). After purification of white blood cells on Ficoll-Hypaque, negative selection of T-CD4⁺ cells, elimination of activated T-CD4 cells (CD25⁺, CD69⁺, HLA-DR⁺) (Milenyi kits), quiescent cells were treated with the compounds for 18-20 h to avoid reinfections. An assay p24 was then performed using a luminescent reagent (Luminata forte, Millipore) at the end of the Innotest. The sensitivity of the test thus modified is ˜0.2 pg p24/ml. The response to BST-1 varied between patients from 0.2 to 80 pg/ml.

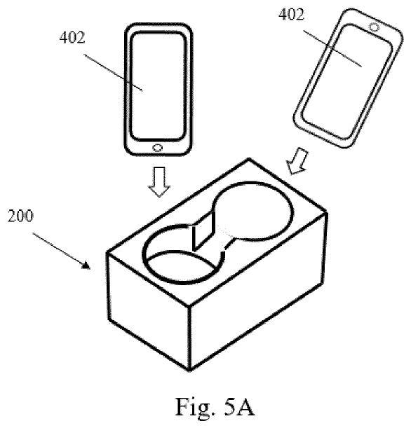

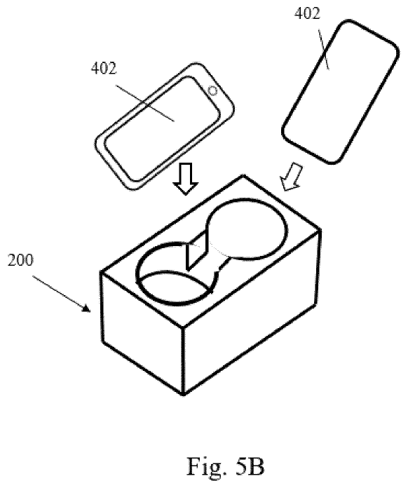

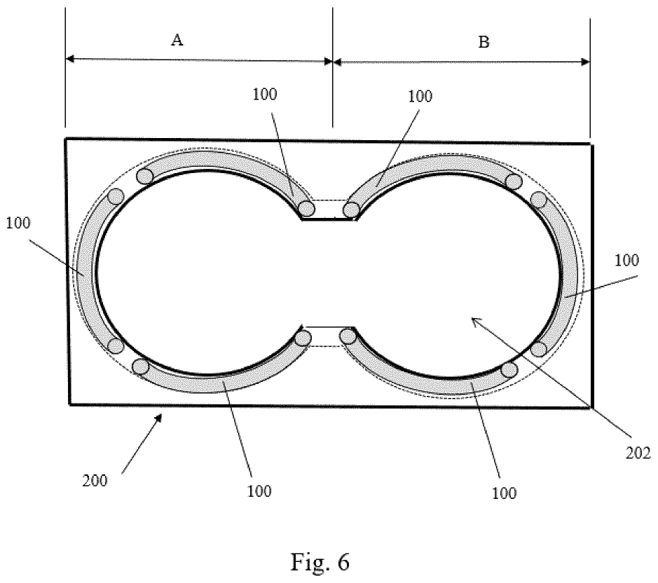

It can be seen (FIG. 5) that the compounds (2) and (3) at 50-100 nM are at least as effective as BST-1 which is considered to be the best LRA currently available.

Effective Doses of the Compound (2)

It has been shown that the compound (2) stabilizes the transcription complex for concentrations of the order of 50-100 nM, effective concentrations which are the same as those for the LRA tests on primary cells. However, in all the tests on laboratory cell lines (HeLa and That), which are immortalized cancer lines, the effective concentrations are of the order of 5 μM. In fact, these lines overexpress drug transporters that can expel (and/or uptake less efficiently) different types of molecules, which could explain the need to use higher concentrations.

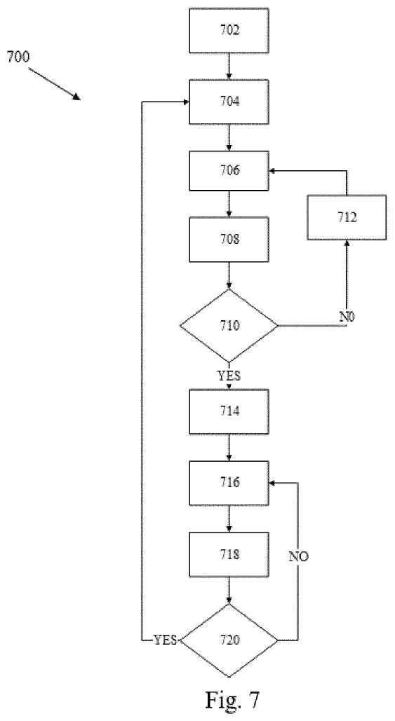

To verify this point, a transactivation test was carried out with primary T-CD4 cells. The result shows that the concentration of the compound (2) most effective in activating transactivation by Tat is 30 nM (FIG. 6), the effective dose being very close to that observed on the latent primaries (50 nM; FIG. 5) and that optimizing the stability of the Tat-TAR complex (80 nM; FIG. 1). The need to use doses 100 times higher on cancerous cells (that is to say 5-10 μM) is due to the cancerization process of these cells.

### CONCLUSION

The compounds according to the invention are effective and safe activators of the Tat protein of HIV-1. These compounds are as effective ex vivo on the cells of HIV⁺ patients as the best LRAs currently available, which target cellular proteins and show numerous side effects. The compounds according to the invention are specific for Tat and more precisely interact with a strong affinity with the viral transcription complex Tat-TAR. A concentration of 30-100 nM is sufficient to ensure maximum effect of these compounds ex vivo.

1-17. (canceled)

18. A method of treating human immunodeficiency virus (HIV) infection in a subject with HIV and/or treating HIV latency in latent HIV-infected cells in a subject infected with HIV comprising administering a compound according to formula (I), or a salt thereof to said subject, 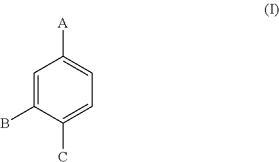
wherein
A represents a heteroaryl optionally substituted by at least one halogen, and
B and C independently represent a radical selected from:
a hydrogen,
a C₁-C₆ alkyl,
a C₁-C₆ alkoxy, and
a group according to formula (II), 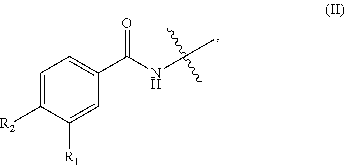
wherein R₁ and R₂ represent, independently of each other, a radical selected from
a hydrogen,
a C₁-C₃ alkyl,
a C₁-C₃ alkoxy,
a group according to the formula —Y—[W]n-Z,
wherein
Y is a radical selected from O, N, S and C;
W is a radical selected from C₁-C₆ alkyl, C₁-C₆ alkylene and C₂-C₆ alkenyl;
n is 0 or 1;
Z represents a phenyl, a pyrrole or a naphthalene, aryl, an imidazole, naphthyl, or a phenanthrene optionally substituted by at least one radical selected from a C₁-C₃ alkyl, a C₁-C₃ alkoxy and a halogen
wherein
when C is hydrogen, methyl or methoxy, B is a group according to formula (II), and conversely when B is hydrogen, methyl or methoxy, C is a group according to formula (II), and
when R₁ is hydrogen, C₁-C₃ alkyl or C₁-C₃ alkoxy, R₂ is the group —Y—[W]n-Z, and conversely when R₂ is hydrogen, C₁-C₃ alkyl or C₁-C₃ alkoxy, R₁ is the group —Y—[W]n-Z.

19. The method according to claim 18, wherein A is selected from a benzimidazole, an imidazo[2,1-b]-1,3,4-thiadiazole, a benzothiazole, a benzoxazolone, a purine Oxazolone, benzodiazole, quinazoline, quinoxaline, oxazolone pyridine, optionally substituted by at least one halogen.

20. The method according to claim 18, wherein A is a benzimidazole optionally substituted by at least one halogen.

21. The method according to claim 20, wherein A is a benzimidazole optionally substituted by a chlorine and/or a bromine.

22. The method according to claim 18, wherein the group —Y—[W]n-Z represents a naphthalenoxy, a pyrolmethoxy, a benzyloxy, a benzene or a phenyloxy, optionally substituted by one or more radicals selected from halogen and C₁-C₃ alkyl.

23. The method according to claim 18, wherein R₁ is methoxy and R₂ is benzyloxy optionally substituted by a halogen.

24. The method according to claim 23, wherein R₂ is optionally substituted with a chlorine.

25. The method according to claim 18, wherein R₁ is hydrogen and R₂ is benzyloxy optionally substituted by a halogen.

26. The method according to claim 25, wherein R₂ is optionally substituted with a chlorine.

27. The method according to claim 18, said compound being selected from 4-(benzyloxy)-N-(5-{imidazo[2,1-b][1,3,4]thiadiazol-2-yl}-2-methoxyphenyl)benzamide, N-[5-(1H-1,3-benzodiazol-2-yl)-2-methylphenyl]-4-[(4-chlorophenyl)methoxy]-3-methoxybenzamide, N-[4-(1H-1,3-benzodiazol-2-yl)phenyl]-3-(benzyloxy)benzamide and N-[3-(1H-1,3-benzodiazol-2-yl)phenyl]-4-[(4-chlorophenyl)methoxy]-3-methoxybenzamide, or salts thereof.

28. The method according to claim 18, wherein said method comprises administering the compound according to formula (I) for the reactivation of HIV in latent HIV-infected cells of said subject.

29. The method according to claim 18, wherein said compound is administered parenterally, enterally or cutaneously.

30. The method according to claim 18, wherein the method further comprises administering to said subject an antiviral agent.

31. The method according to claim 18, said method being a method for treating HIV infection in a subject with HIV having latent infected cells.

32. The method according to claim 18, said method being a method for treating HIV latency in latent HIV-infected cells in a subject with HIV.

33. A pharmaceutical composition comprising a pharmaceutically acceptable carrier and a compound of formula (I) 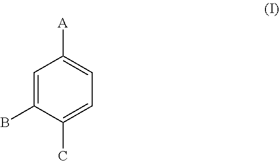
wherein
A represents a heteroaryl optionally substituted by at least one halogen, and
B and C independently represent a radical selected from:
a hydrogen,
a C₁-C₆ alkyl,
a C₁-C₆ alkoxy, and
a group according to formula (II), 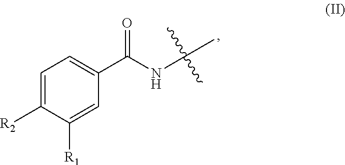
wherein R₁ and R₂ represent, independently of each other, a radical selected from
a hydrogen,
a C₁-C₃ alkyl,
a C₁-C₃ alkoxy,
a group according to the formula —Y—[W]n-Z,
wherein
Y is a radical selected from O, N, S and C;
W is a radical selected from C₁-C₆ alkyl, C₁-C₆ alkylene and C₂-C₆ alkenyl;
n is 0 or 1;
Z represents a phenyl, a pyrrole or a naphthalene, aryl, an imidazole, naphthyl, or a phenanthrene optionally substituted by at least one radical selected from a C₁-C₃ alkyl, a C₁-C₃ alkoxy and a halogen
wherein
when C is hydrogen, methyl or methoxy, B is a group according to formula (II), and conversely when B is hydrogen, methyl or methoxy, C is a group according to formula (II), and
when R₁ is hydrogen, C₁-C₃ alkyl or C₁-C₃ alkoxy, R₂ is the group —Y—[W]n-Z, and conversely when R₂ is hydrogen, C₁-C₃ alkyl or C₁-C₃ alkoxy, R₁ is the group —Y—[W]n-Z.

34. A method for the reactivation of HIV in latent HIV-infected cells comprising contacting latent HIV-infected cells with a composition according to claim 33.
